(19) United States
(12) Patent Application Publication
(10) Pub. No.: US 2003/0120237 A1
(43) Pub. Date: Jun. 26, 2003

(54) Absorbent structure, and absorbent article containing the absorbent structure

(76) Inventor: Ted Guidotti, Goteborg (SE)

Correspondence Address: BURNS, DOANE, SWECKER & MATHIS, L.L.P., P.O. Box 1404, Alexandria, VA, 22313-1404, US

(21) Appl. No.: 10/323,665

(22) Filed: Dec. 20, 2002

Related U.S. Application Data
(60) Provisional application No. 60/341,849, filed on Dec. 21, 2001.

(51) Int. Cl. A61F 13/15; A61F 13/20

(52) U.S. Cl. 604/380; 604/378; 604/368

(57) ABSTRACT

An absorbent structure (3) for an absorbent article has, in the longitudinal direction, a central portion (11) and two end portions (12, 13). The average density of the material in the central portion (11), at least in the dry state, is higher than the average density of the material in the end portions (12, 13), and in that the central portion (11), at least in the dry state, has a lower basis weight than the end portions (12, 13), the density and the basis weight being calculated on the basis of the entire surface area of the central portion (11) or as appropriate the entire surface area of the end portion (12, 13).

### CROSS-REFERENCE TO RELATED APPLICATIONS

The present application claims the benefit of U.S. Provisional Application No. 60/341,849, filed in the United States on Dec. 21, 2001, the entire contents of which are hereby incorporated herein by reference.

### BACKGROUND OF THE APPLICATION 1. Technical Field

The present invention relates to an absorbent structure for an absorbent article such as a diaper, an incontinence pad, a sanitary towel or the like, the absorbent structure having, in the longitudinal direction, a central portion and two end portions. The invention also relates to an absorbent article containing such an absorbent structure.

2. Background Art

Absorbent structures for disposable absorbent articles such as diapers, incontinence pads and sanitary towels are in most cases constructed from one or more layers of hydrophilic fibers, for example, cellulose fluff pulp. Also included in most cases are superabsorbents, which are polymers which are capable of absorbing liquid corresponding to many times their own weight. The absorbent structure can also comprise further parts, for example, for improving liquid-spreading or for increasing the cohesive capacity and the capacity for resisting deformation under loading.

A problem in the use of baby diapers and incontinence pads for adults, which have to be capable of receiving and absorbing large quantities of liquid in a relatively short time, is that these often leak before the total absorption capacity has been fully utilized. As a large quantity of liquid can be discharged in a few seconds, this can lead to the absorbent structure temporarily becoming locally saturated with liquid in what is known as the liquid-receiving zone. There may not be enough time for the liquid to spread further to other parts of the absorbent structure, which can in turn lead to liquid leaking out of the diaper.

From EP 0,254,476 and EP 0,325,416, an absorbent structure is known, which, in a zone essentially directly in front of the wetting area, has a lower basis weight and a lower density compared with surrounding portions of the absorbent structure. By virtue of such a structure, an area with an open structure and a great liquid take-up capacity is obtained. However, it can be difficult for the liquid to spread from the high-bulk open structure in the wetting area to the two end portions.

From EP 0,525,778, an absorbent structure is also known, which, in a zone essentially directly in front of the wetting area, has a higher basis weight and a lower density than in the end portions. One advantage of such a structure is that the wetting area has a high-bulk open structure which is capable of rapidly receiving a large quantity of liquid.

It has nevertheless been found that there is room for improvement with regard to the construction of an absorbent structure which is capable of rapidly receiving a large quantity of liquid at the same time as it is thin, flexible and discreet to wear.

### SUMMARY

One embodiment of the present invention includes an absorbent structure for an absorbent article of the type mentioned in the introduction. The absorbent structure is capable of rapidly receiving a large quantity of liquid at the same time as it is thin, flexible and discreet to wear.

In the embodiment of the invention, the average density of the material in the central portion, at least in the dry state, is higher than the average density of the material in the end portions, and the central portion, at least in the dry state, has a lower basis weight than the end portions, the density and the basis weight being calculated on the basis of the entire surface area of the central portion or as appropriate the entire surface area of the end portions.

As the central portion constitutes the area of the article which receives the discharged liquid first, this area should preferably rapidly receive a large quantity of liquid. Furthermore, the central portion is subjected to relatively greater mechanical stress than the end portions during use of the absorbent article. This means that it is especially preferable for a thin, soft and flexible material to be positioned in the central portion of the absorbent structure. The central portion means that area in the absorbent structure which is located in what is known as the liquid-receiving zone, that is to say the zone which is expected to be wetted first by the discharged liquid when the absorbent structure is used in an absorbent article. The liquid-receiving zone is usually located between 10 cm and 20 cm from the front edge of the absorbent structure.

According to one embodiment, the absorbent structure comprises a liquid-storing layer and a liquid-receiving layer, the liquid-receiving layer being positioned at least in the central portion of the absorbent structure. The upper, liquid-receiving layer is preferably such that it expands rapidly in the thickness direction of the structure on wetting. One advantage of a liquid-receiving layer which expands in the thickness direction of the structure on wetting is that such a layer is thin before wetting and that it expands rapidly in the thickness direction on wetting. This means that such a structure becomes more open and voluminous and can receive a large quantity of liquid in a short time. It is also possible to have a liquid-receiving layer which has a very narrow central portion before wetting but which, on wetting, expands in the direction of its plane so that its width increases. On wetting, the liquid-receiving layer can therefore expand both in the thickness direction of the structure and in the direction of its plane, or alternatively in only one of said directions.

One example of a material for the liquid-receiving layer is a superabsorbent foam. Such a superabsorbent foam preferably has a density in the dry state of from 0.2 g/cm³ to 1.0 g/cm³, and a basis weight in the dry state of from 200 g/m² to 800 g/m². One advantage of a superabsorbent foam is that it is a soft and flexible material. When a wearer moves, the article is subjected to the greatest mechanical stress in the crotch portion of the article, which means that it is especially advantageous for a soft and flexible material to be positioned in the central portion of the absorbent structure. Another advantage of a superabsorbent foam as liquid-receiving layer is that such a material is also capable of absorbing and storing liquid, which in turn reduces the risk of liquid leakage. The superabsorbent foam is preferably hard-compressed in the dry state, expanding in the thickness direction of the structure on wetting. It is also possible to have a superabsorbent foam which has a very narrow central portion before wetting but which, on wetting, expands in the direction of its plane so that its width increases. On wetting, the superabsorbent foam can therefore expand both in the thickness direction of the structure and in the direction of its plane, or alternatively in only one of said directions.

Another example of a suitable material for the liquid-receiving layer is a foam made of regenerated cellulose, what is known as a viscose foam. Such a viscose foam is preferably hard-compressed in the dry state, expanding in the thickness direction of the structure and/or in the direction of its plane on wetting. A further example of a material for the liquid-receiving layer is a dry-formed sheet containing 5-100% cellulose fibers, which has a density of from 0.2 g/cm³ to 1.0 g/cm³ and a basis weight of from 30 g/m² to 1200 g/m². Such a dry-formed sheet can be formed by compression of a web containing cellulose fibers without subsequent defibration and fluff-formation.

The liquid-storing layer is preferably a mixed structure made of cellulose fluff fibers and superabsorbent material. The mixed structure comprising cellulose fluff fibers and superabsorbent material preferably has a density in the dry state of from 0.1 g/cm³ to 0.7 g/cm³ and a basis weight in the dry state of from 200 g/m² to 1500 g/m². The superabsorbent material can be in the form of, for example, particles, flakes or fibers. However, a liquid-storing layer comprising only a superabsorbent material, for example a superabsorbent foam, is also conceivable.

According to another embodiment, the absorbent structure also comprises a liquid-spreading layer. The liquid-spreading layer comprises a thin layer which is positioned furthest away from the side which, when the absorbent structure is arranged in an absorbent article, is intended to face the wearer, and extends over the whole surface of the absorbent structure. The liquid-spreading layer comprises a material with a good liquid-spreading capacity, for example, a thin layer of chemically produced cellulose fluff fibers. Chemically produced fluff pulps, which comprise fine fibers of essentially pure cellulose, generally have a good liquid-spreading capacity. In order to obtain a good liquid-spreading capacity, such a liquid-spreading layer preferably comprises a structure with fine capillaries, and such a layer preferably has a high density. If appropriate, such a cellulose-based liquid-spreading layer also contains a small quantity of superabsorbent material in the form of, for example, particles. One advantage of a liquid-spreading layer is that such a layer is able to spread the liquid coming from the liquid-receiving layer in the central portion via the liquid-spreading layer out to the two end portions where the liquid can be taken up and stored in the liquid-storing layer. Another suitable material for the liquid-spreading layer is a thin layer of HIPE (high internal phase emulsion) foam. Such a foam is produced by polymerization of a water-in-oil emulsion. The solid phase of the foam creates a capillary system.

Another embodiment of the invention includes an absorbent article such as a diaper, an incontinence pad, a sanitary towel or the like, which has an essentially oblong shape. The article comprises a liquid-permeable surface layer, a liquid-impermeable backing layer, and, enclosed between these layers, an absorbent structure according to any one of the embodiments described.

### BRIEF DESCRIPTION OF THE FIGURES

The invention will be described in greater detail below with reference to the illustrative embodiments shown in the accompanying drawings, in which

FIG. 1 shows a diaper seen from the side which is intended to face the wearer during use;

FIG. 2 shows the absorbent structure in the diaper according to FIG. 1 from the side which is intended to face the wearer during use;

FIG. 3 shows a section through the absorbent structure along the line III-III in FIG. 2;

FIG. 4 shows an alternative absorbent structure seen from the side which is intended to face the wearer, and

FIG. 5 shows a section through the absorbent structure along the line V-V in FIG. 4.

### DETAILED DESCRIPTION OF THE PREFERRED EMBODIMENTS

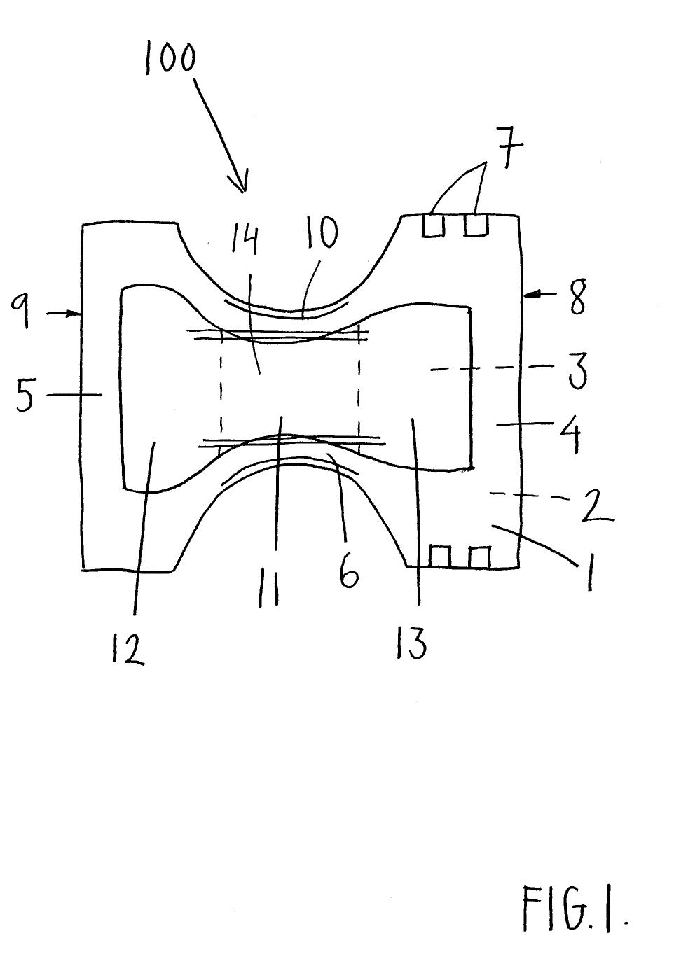

The diaper 100 shown in FIG. 1 comprises a liquid-permeable surface layer 1, for example made of non-woven fabric or perforated plastic film, a liquidtight backing layer 2, for example made of plastic film or hydrophobic non-woven fabric, and an absorbent structure 3 enclosed between the two layers 1 and 2.

The diaper 100 is intended to surround the lower part of the abdomen of a wearer like a pair of absorbent pants. It has a rear portion 4 intended to face backwards on the wearer during use of the diaper, a front portion 5 intended to face forwards on the wearer during use, and, located between the rear portion 4 and the front portion 5, a narrower crotch portion 6 which is intended to be arranged in the crotch of the wearer between the legs of the latter. In order for it to be possible for the diaper to be fastened together to form the desired pants-shape, tape flaps 7 are arranged close to the rear waist edge 8 of the diaper. During use, the tape flaps 7 are attached to the outside of the front portion 5 of the diaper, close to the front waist edge 9, the diaper then being held together around the waist of the wearer. Other fastening means, such as touch-and-close tape, hooks or the like, are of course also possible.

The diaper 100 according to FIG. 1 also comprises pretensioned elastic elements 10 which can consist of suitable material, such as elastic foam, elastic bands or thread-covered threads. For the sake of simplicity, these elements have been shown in the extended state in FIG. 1. As soon as the stretching ceases, however, the elastic elements contract and in this way form elastic legbands of the diaper.

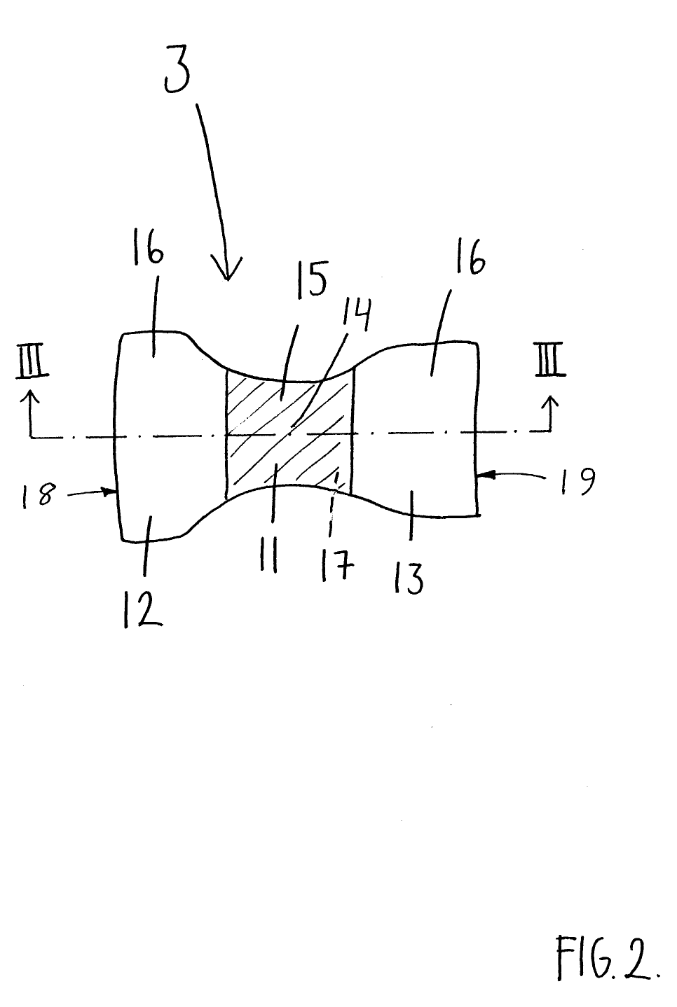

The diaper 100 also comprises an absorbent structure 3. The absorbent structure 3 has a central portion 11 positioned in the crotch portion 6 of the diaper and two end portions 12, 13 positioned in the front portion 5 and, respectively, the rear portion 4 of the diaper and is shown in detail and different alternative embodiments in FIGS. 2-5.

Alternatively, the liquid-permeable surface layer 1 can be a part of the absorbent structure 3. One advantage of such a construction is that, when the absorbent structure 3 comprises material which is hard-compressed in the dry state but which expands rapidly on wetting, the absorbent structure 3 can absorb freely without being hindered by the risk that the enclosed space between the liquidtight backing layer 2 and the liquid-permeable surface material 1 may be too small.

FIG. 2 shows the absorbent structure 3 in the diaper 100 according to FIG. 1 from the side which is intended to face the wearer during use. The absorbent structure 3 has a central portion 11, a first end portion 12 and a second end portion 13. The absorbent structure also has a front end edge 18 and a rear end edge 19. The central portion 11 means that area in the absorbent structure which is located in what is known as the liquid-receiving zone 14, that it to say the zone which is expected to be wetted first by the discharged liquid when the absorbent structure 3 is used in an absorbent article. The liquid-receiving zone is usually located between 10 cm and 20 cm from the front edge 18 of the absorbent structure 3.

The absorbent structure 3 is furthermore constructed from three different layers, a liquid-receiving layer 15, a liquid-storing layer 16 and a liquid-spreading layer 17. The liquid-receiving layer 15 is positioned in the central portion 11 of the absorbent structure and is intended to lie next to the liquid-permeable surface layer 1 when used in an absorbent article. The liquid-receiving layer 15 is made from, for example, a superabsorbent foam which is hard-compressed in the dry state but which expands rapidly in the thickness direction of the liquid-receiving layer 15 on contact with urine. The liquid-receiving layer 15 can alternatively be a cellulose-based foam. An example of a cellulose-based foam is a compressed foam made of regenerated cellulose, what is known as a viscose foam. On wetting, such a compressed viscose foam expands very rapidly in the thickness direction of the material. It is also possible for the liquid-receiving layer 15 to have a very narrow central portion before wetting, but for the layer, on wetting, to expand in the direction of its plane so that its width increases. On wetting, the liquid-receiving layer 15 can therefore expand both in the thickness direction of the structure and in the direction of its plane, or alternatively in only one of said directions. Furthermore, the liquid-receiving layer 15 can be a dry-formed sheet containing 5-100% cellulose fibers, which has a density of from 0.2 to 1.0 g/cm³ and a basis weight of from 30 to 1200 g/m², which sheet has been formed by compression of a web containing cellulose fibers without subsequent defibration and fluff-formation.

The liquid-storing layer 16 is positioned in the end portions 12, 13 of the absorbent structure 3. The liquid-storing layer 16 in the absorbent structure 3 in FIG. 2 consists of two separate units, one liquid-storing unit being positioned in the first end portion 12 and the other liquid-storing unit being positioned in the second end portion 13. The liquid-storing layer 16 usually contains superabsorbent material, that is to say material which is capable of absorbing liquid corresponding to many times its own weight, and which material is also capable of retaining liquid under external loading. The liquid-storing layer is preferably a mixed structure made of cellulose fluff fibers and superabsorbent material. The mixed structure consisting of cellulose fluff fibers and superabsorbent material preferably has a density in the dry state of from 0.1 g/cm³ to 0.7 g/cm³ and a basis weight in the dry state of from 200 g/m² to 1500 g/m². The superabsorbent material can be in the form of, for example, particles, flakes or fibers. However, a liquid-storing layer consisting of only a superabsorbent material, for example a superabsorbent foam, is also conceivable.

The liquid-spreading layer 17 comprises a thin layer which is positioned furthest away from the side which is intended to face the wearer, and extends over the whole surface of the absorbent structure. The liquid-spreading layer 17 comprises a material with a good liquid-spreading capacity, for example a thin layer of chemically produced cellulose fluff fibers. Chemically produced fluff pulps, which comprise fine fibers essentially pure cellulose, generally have a good liquid-spreading capacity. In order to obtain a good liquid-spreading capacity, such a liquid-spreading layer comprises a structure with fine capillaries, and such a layer preferably has a high density. If appropriate, a cellulose-based liquid-spreading layer also contains a small quantity of superabsorbent material in the form of, for example, particles. One advantage of a liquid-spreading layer 17 is that such a layer is able to spread the liquid coming from the liquid-receiving layer 15 in the central portion 11 via the liquid-spreading layer 17 out to the two end portions 12, 13 where the liquid can be taken up and stored in the liquid-storing layer 16. Another suitable material for the liquid-spreading layer 17 is a thin layer of HIPE (high internal phase emulsion) foam. Such a foam is produced by polymerization of a water-in-oil emulsion. The solid phase of the foam creates a capillary system.

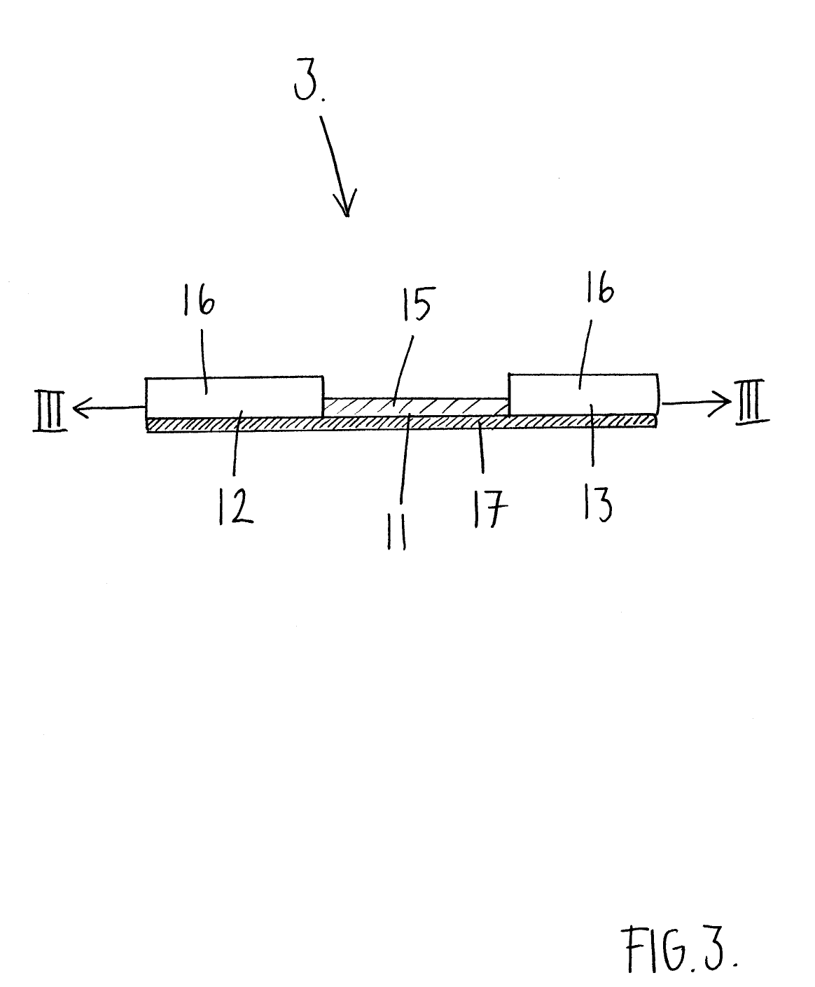

FIG. 3 shows a longitudinal section through the absorbent structure 3 along the line III-III in FIG. 2. The absorbent structure therefore has a central portion 11, a first end portion 12 and a second end portion 13. As described in FIG. 2, the absorbent structure 3 is constructed from three different layers, a liquid-receiving layer 15, a liquid-storing layer 16 and a liquid-spreading layer 17. FIG. 3, which shows a section through the absorbent structure 3, shows that, in the dry state, the liquid-receiving layer 15 in the central portion 11 is thinner than the liquid-storing layer 16 in the two end portions 12, 13. By virtue of the liquid-receiving layer 15 being thinner than the liquid-storing layer 16, a lower basis weight is obtained for the material in the central portion 11 than for the material in the end portions 12, 13 in spite of that fact that the average density of the material in the central portion 11 is higher than the average density of the material in the end portions 12, 13. In this way, an absorbent structure 3 is obtained which, in an absorbent article, is discreet and comfortable to wear at the same time as it has good liquid-receiving, liquid-spreading and liquid-storing capacity.

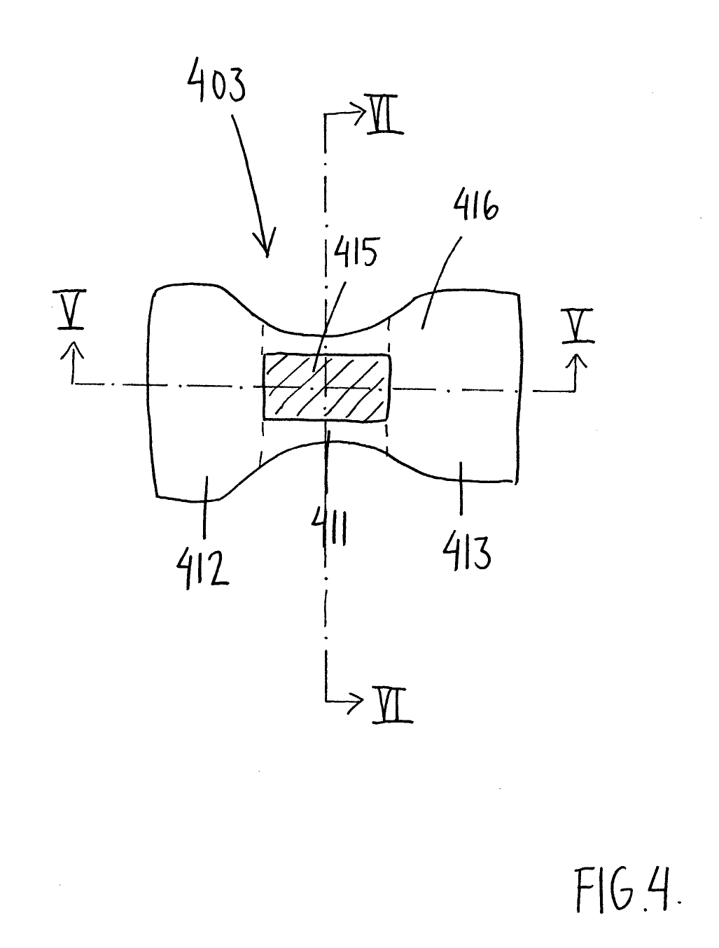

FIG. 4 shows an alternative embodiment of an absorbent structure 403 according to the invention seen from the side which is intended to face the wearer. The absorbent structure has a longitudinal direction V and a transverse direction VI.

The absorbent structure 403 also has a central portion 411, a first end portion 412 and a second end portion 413. Furthermore, the absorbent structure 403 is constructed from two different layers, a liquid-receiving layer 415 and a liquid-storing layer 416. The liquid-receiving layer 415 is positioned in the central portion 411 of the absorbent structure and is intended to be positioned next to the liquid-permeable surface layer when used in an absorbent article. The liquid-storing layer 416 extends over the whole surface of the absorbent structure 403. The liquid-storing layer 416 is intended to be positioned next to the liquidtight backing layer when the absorbent structure is used in an absorbent article. In the central portion 411 of the absorbent structure, the liquid-storing layer is slightly wider in the transverse direction VI of the absorbent structure than the liquid-receiving layer 415. In the central portion 411 of the absorbent structure, the liquid-receiving layer 415 is therefore positioned in what is known as the wetting zone, that is to say in the area which is expected to be wetted first by the discharged bodily fluid. The liquid-receiving layer 415 and the liquid-storing layer 416 can of course also have the same extent in the transverse direction VI, that is to say have the same width.

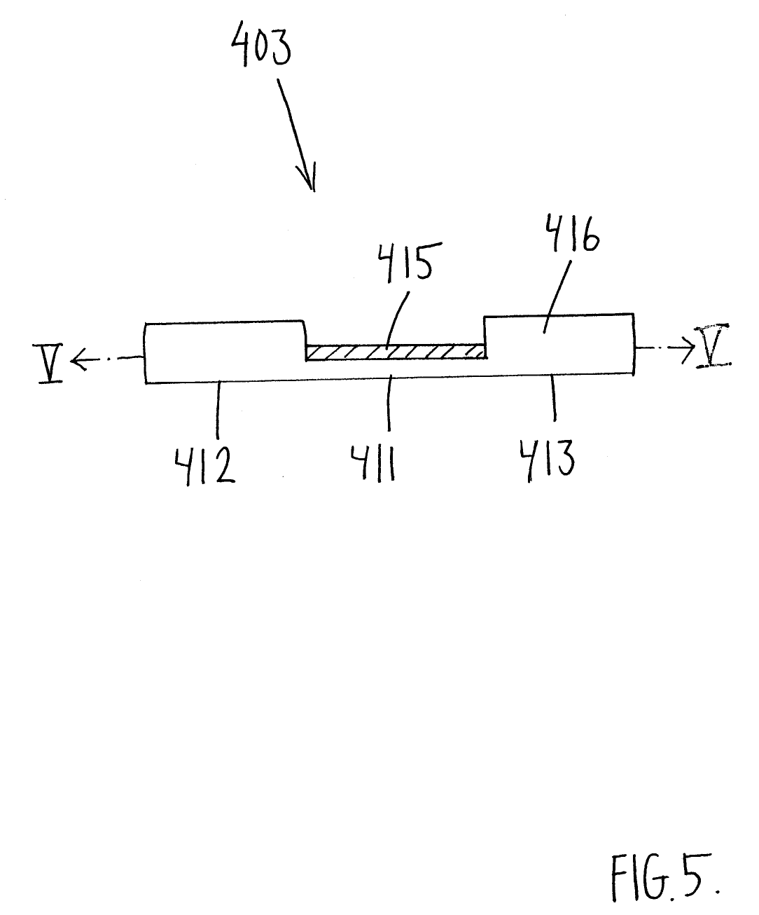

FIG. 5 shows a longitudinal section through the absorbent structure along the line V-V in FIG. 4, that is to say a section through the absorbent structure in its longitudinal direction. FIG. 5 therefore shows the central portion 411 and the two end portions 412, 413. The two end portions 412, 413 therefore consist of only the liquid-storing layer 416, while the central portion 411 also comprises the liquid-receiving layer 415. It can be seen from FIG. 5 that the thickness of the liquid-storing layer 416 is greater in the two end portions 412, 413 than in the central portion 411. As FIG. 5 also shows, the total thickness of the absorbent structure in the two end portions is greater than the total thickness of the absorbent structure in the central portion 411. By virtue of the absorbent structure 403 being thinner in the central portion 411 than in the two end portions 412, 413, a lower basis weight is obtained for the material in the central portion 411 than in the end portions 412, 413 in spite of that fact that the average density of the material in the central portion 411 is higher than the average density of the material in the end portions 412, 413. It is also possible for the absorbent structure shown in FIGS. 4 and 5 to comprise a thin liquid-spreading layer which is located against the liquid-storing layer 416 and is therefore positioned next to the liquidtight backing layer when the absorbent structure is used in an absorbent article.

The diaper 100 described in FIG. 1 and the absorbent structures described in FIGS. 2-5 constitute only a few illustrative embodiments. Therefore, the shape of the diaper as also its design in other respects can be varied, and the shape of the absorbent structure as also its design in other respects can be varied.

1. An absorbent structure for an absorbent article, the absorbent structure comprising, in the longitudinal direction, a central portion made of a first material and two end portions made of a second material,
an average density of the first material in the central portion, at least in a dry state, is higher than an average density of the second material in the end portions, and
the central portion, at least in the dry state, has a lower basis weight than the end portions.

2. The absorbent structure according to claim 1, wherein the density and the basis weight are calculated on the basis of the entire surface area of the central portion or as appropriate the entire surface area of the end portions.

3. The absorbent structure according to claim 2, wherein the density and the basis weight are calculated on the basis of the entire surface area of the end portions.

4. The absorbent structure according to claim 1, wherein the density and the basis weight are calculated on the basis of the entire surface area of the end portions.

5. The absorbent structure according to claim 2, wherein the absorbent article is a diaper, an incontinence pad, or a sanitary towel.

6. The absorbent structure according to claim 1, wherein the absorbent structure comprises a liquid-storing layer and a liquid-receiving layer, the liquid-receiving layer being positioned at least in the central portion of the absorbent structure.

7. The absorbent structure according to claim 6, wherein the liquid-receiving layer is a superabsorbent foam, the superabsorbent foam having a density in the dry state of from 0.2 g/cm³ to 1.0 g/cm³, and a basis weight in the dry state of from 200 g/m² to 800 g/m².

8. The absorbent structure according to claim 6, wherein the liquid-receiving layer is a foam made of regenerated cellulose.

9. The absorbent structure according to claim 6, wherein the liquid-receiving layer is a dry-formed sheet containing 5-100% cellulose fibres, which has a density of from 0.2 g/cm³ to 1.0 g/cm³ and a basis weight of from 30 g/m² to 1200 g/m², which sheet has been formed by compression of a web containing cellulose fibres without subsequent defibration and fluff-formation.

10. The absorbent structure according to claim 6, wherein the liquid-receiving layer expands in the thickness direction of the absorbent structure on wetting.

11. The absorbent structure according to claim 6, wherein the liquid-storing layer is a mixed structure made of cellulose fluff fibres and superabsorbent material.

12. The absorbent structure according to claim 7, wherein the liquid-storing layer has a density in the dry state of from 0.1 g/cm³ to 0.7 g/cm³ and a basis weight in the dry state of from 200 g/m² to 1500 g/m².

13. The absorbent structure according to claim 1, wherein the absorbent structure comprises a liquid-spreading layer.

14. An absorbent article, which has an essentially oblong shape and comprises a liquid-permeable surface layer, a liquidtight backing layer, and, enclosed between these layers, an absorbent structure, which absorbent structure has, in the longitudinal direction, a central portion and two end portions, and an average density of a material in the central portion, at least in the dry state, is higher than an average density of a material in the end portions, and the central portion, at least in the dry state, has a lower basis weight than the end portions, the basis weight and the density being calculated on the basis of the entire surface area of the central portion and the entire surface area of the end portions.

15. The absorbent structure according to claim 14, wherein the absorbent article is a diaper, an incontinence pad, or a sanitary towel.

16. An absorbent structure for an absorbent article, the absorbent structure comprising:
a liquid spreading layer;
a liquid receiving layer made of a first material is mounted to one side of the liquid spreading layer in a central portion thereof;
two liquid storing sections made of a second material are mounted on the one side of the liquid spreading layer on opposite end portions of the liquid spreading layer, wherein the liquid receiving layer is located between the two liquid storing sections;
an average density of the first material of the liquid receiving layer, at least in a dry state, is higher than an average density of the second material of the two liquid storing sections; and
wherein the absorbent structure is thinner in the central portion where the liquid receiving layer is located than in the end portions where the liquid storing sections are located.

17. The absorbent structure according to claim 16, wherein the absorbent article is a diaper, an incontinence pad, or a sanitary towel.

18. The absorbent structure according to claim 16, wherein the liquid receiving layer has, at least in a dry state, a lower basis weight than the liquid storing sections.
